(19) United States
(12) Patent Application Publication
(10) Pub. No.: US 2004/0146587 A1
(43) Pub. Date: Jul. 29, 2004

(54) Topical composition for relief of pain and minor skin irritations

(76) Inventor: James Spicer, Orlando, FL (US)

Correspondence Address: Elsie C. Turner, Suite 204, 201 Park Place, Altamonte Springs, FL, 32701, US

(21) Appl. No.: 10/352,428

(22) Filed: Jan. 29, 2003

(51) Int. Cl. A61K 35/78

(52) U.S. Cl. 424/736; 424/744; 424/756

(57) ABSTRACT

The present invention provides a composition of naturally-occurring agents for external application to a human body for relief of muscular and joint aches and pains, congestion, and skin irritations, including burns, sunburn and insect bites, comprising an aqueous reduction of effective amounts of aloe vera, selected citrus peelings, and ginger root in an emollient cream carrier.

### BACKGROUND

The present invention is directed to an analgesic composition, safe for oral ingestion as well as topical application, found to relieve muscle and joint aches and pains, respiratory congestion, insect bites and stings, and burns. The active ingredients are naturally occurring and comprise an aqueous extract of aloe vera, citrus peel, and ginger root in an emollient cream. The composition is applied topically and rubbed into the skin.

It is widely known that aloe vera can relieve burns to the skin, including second-degree burns, and many compositions containing aloe vera can be found in the marketplace. U.S. Pat. No. 5,958,418 to Prillerman is for one such composition and method intended for topical application for the relief of muscular pain. The Prillerman composition contains aloe vera, goldenseal, comfrey root and capsicum. Capsicum is known to relieve pain by stimulating the production of heat in the skin and underlying flesh. Capsicum, however, is a powerful irritant, especially to the eyes, lips and mucous membranes, and the basis of repellant sprays to ward off attackers, and is not known for healing properties.

International Publication No. WO 91/16914, a patent application by Bockow et al., discloses the use of aloe as a deodorizing agent for oils of marine animals. These oils, characterized by unpleasant odor, are intended for use as anti-inflammatory agents, analgesics, vasodilatory agents, anti-pruritic agents, anesthetics, antiseptics, and other similar compositions.

A product made by B. F. Ascher, Lenexa, KS, called Pain-X, an analgesic gel, purporting to contain aloe vera to soothe arthritis and muscle pain, was announced in New Product News, Sep. 10, 1997.

The use of ginger root and citrus peels in combination with aloe vera as a therapeutic topical cream or salve has not been known heretofore or suggested in any known publication. Individually, each of these ingredients has known beneficial properties. Ginger compresses have been used as a home or folk remedy on cysts and tumors (www.goodhealthinfo.net/mdr/home_remedies.htm). Ginger has long been ingested in various forms, including ginger ale, as a soothing agent for irritations of the gastrointestinal tract. Citrus peels contain oils and limonene, which are natural degreasers, cleaners, deodorizers, and emulsifiers. It has been reported in the Journal of Medicinal Food (Vol 1, No. 2) that limonene in an oral combination with the anti-cholesterol drug lovastatin has been found to inhibit the growth of cancer. Limonene, a terpene, has also been used in shampoo for dogs to soothe dermatitis caused by flea bites. There are also claims that limonene is an effective antibacterial agent against staphyloccus and streptococcus, and that it kills fleas, aphids, mites, fire ants, houseflies, stable flies, black soldier flies, paper wasps and house crickets. A product sold by Koster Keunen, Inc., called Orange Wax, a bi-product of orange peels from the food industry, claims to be sunscreen-enhancing, anti-oxidant, anti-microbial, and anti-inflammatory.

There are no products known to the Applicant that contain extracts of aloe vera, ginger, and citrus peel.

Accordingly, it has been an object of this invention to provide an improved analgesic balm or cream having pain-relieving and curative powers for muscle and joint aches and pains and surface irritations of the skin, such as burns and insect bites, combining the curative and pain-relieving properties of the naturally-occurring, safe, and ingestible ingredients of aloe vera, citrus peels, and ginger.

### SUMMARY OF THE INVENTION

The present invention provides a composition of naturally-occurring agents for external application to a human body for relief of muscular and joint aches and pains, congestion, and skin irritations, including burns, sunburn and insect bites, comprising an aqueous extract of effective amounts of aloe vera, selected citrus peelings, and ginger root in an emollient cream carrier.

The present invention further discloses a process for the preparation of a composition formulated as a skin cream or balm suitable for external application to the human body for relieving muscular and joint aches and pains, congestion, and skin irritations comprising:

a.) mixing effective amounts of aloe vera plant and roots, ginger root, selected citrus peels and water;

b.) boiling the mixture until its volume is substantially reduced; and

c.) blending approximately equal parts of the reduced mixture and cream emollient.

### DETAILED DESCRIPTION OF THE PREFERRED EMBODIMENT

The components of the present invention are naturally-occurring, edible plant materials for topical application to mammals, including human beings, to aching limbs and joints, insect bites, bee stings, and in general to areas of inflammation.

The active ingredients utilized in the present invention include aqueous blended mixture of aloe vera plant, ginger root, and orange, lemon and grapefruit peels. Ginger root and citrus fruits are commonly available in grocery stores, and live aloe vera plants are commonly sold in plant nurseries and garden stores.

There are hundreds of varieties of aloe, all part of the lily family, but the variety that is most valuable nutritionally and that should be used in this invention is aloe vera barbadensis miller.

The preferred effective amounts of the active ingredients used to create the combination, before being added to water, are: 23.9% aloe vera plant (roots and all), 23.9% orange peel, 23.9% lemon peel, 23.9% grapefruit peel, and 5.4% ginger root. One gallon of aqueous extract of these ingredients is made by combining two gallons of water with two pounds each of aloe vera, orange peel, lemon peel, and grapefruit peel, and six ounces of ginger root. The combination is then boiled until its volume is reduced to one gallon. The resulting reduction is then processed in a mechanical food processor or blender to macerate and blend the mixture into a smooth “starter”.

The above-stated percentages of active ingredients may be varied without significantly reducing the effectiveness of the resulting product. For instance, the amounts of the aloe vera plant and each of the citrus peels may range from 20% to 30% of the combination, and the amount of ginger may vary from 3% to 8%.

The preferred amounts of blended mixture and emollient cream are: equal parts of the mixture and cream emollient. This ratio could vary between 2:1 or 1:2.

The composition of this invention is specifically directed to a topical application. The composition is applied to the affected area of skin and massaged or rubbed until absorbed. The composition has been proven more effective than aloe vera alone to relieve the pain of arthritis, muscle aches, insect bites, stings, and burns, and to relieve congestion when applied to the chest or sinuses.

Changes and modifications in the specifically described embodiments can be made without departing from the scope of the invention, which is intended to be limited only by the scope of the appended claims.

I claim:

1. An analgesic composition for topical application for the relief of muscle and joint aches and pains, respiratory congestion, insect bites and stings, and burns including sunburn, comprising:
an emollient carrier cream; and
a blended reduction of plant ingredients, namely, aloe plant, selected citrus peels and ginger root in water.

2. The composition according to claim 1 wherein said blended reduction comprises about 1 pound each of aloe plant with roots, orange peel, grapefruit peel, and lemon peel, and about 3 ounces of ginger root, per gallon of water, boiled together until the volume thereof is reduced by half.

3. The composition according to claim 2 wherein the composition is comprised of equal parts of emollient carrier cream and blended reduction

4. The composition according to claim 3, wherein said blended reduction has a smooth consistency.

5. The composition according to claim 4 wherein said plant ingredients comprise by weight:
aloe vera plant and roots, 20-30%;
orange peel, 20-30%;
lemon peel, 20-30%;
grapefruit peel, 20-30%; and
ginger root, 3-9%.

6. The composition according to claim 5 wherein said plant ingredients comprise by weight:
aloe vera plant and roots, 23.9%;
orange peel, 23.9%;
lemon peel, 23.9%
grapefruit peel, 23.9%; and
ginger root. 5.4%.

7. An analgesic composition for topical application for the relief of muscle and joint aches and pains, respiratory congestion, insect bites and stings, and burns including sunburn, comprising equal parts of an emollient carrier cream and a blended reduction by half of a mixture of selected plant ingredients in water, wherein the ratio of plant ingredients to water in said mixture is four pounds three ounces per gallon, said plant ingredients comprising by weight:
aloe vera plant with roots, 23.9%;
orange peel, 23.9%;
lemon peel, 23.9%
grapefruit peel, 23,9%; and
ginger root. 5.4 %.

8. The composition according to claim 7 wherein it has a smooth consistency.
